(19) United States
(12) Patent Application Publication
(10) Pub. No.: US 2015/0352038 A1
(43) Pub. Date: Dec. 10, 2015

(54) A DOSAGE FORM FOR ADMINISTERING AN ACTIVE PRINCIPLE FOR ACCELERATED SLEEP INDUCTION AND/OR FOR TREATING SLEEP DISORDERS AND/OR FOR TREATING A CENTRAL NERVOUS SYSTEM DISORDER

(71) Applicants: Philippe PEROVITCH, (US); Jack AUZERIE, (US); Raphael ROSSELLO, Paris (FR)

(72) Inventors: Philippe PEROVITCH, Le Temple (FR); Jack AUZERIE, Sainte-Eulalie (FR)

(73) Assignee: Raphael ROSSELLO, Paris (FR)

(21) Appl. No.: 14/759,822

(22) PCT Filed: Jan. 14, 2014
(86) PCT No.: PCT/FR2014/050070

§ 371 Date: Jul. 8, 2015

(30) Foreign Application Priority Data
Jan. 14, 2013 (FR) 1350308

(51) Int. Cl. A61K 9/00; A61K 47/10; A61K 31/437; A61K 31/4985; A61K 31/519; A61K 31/165; A61K 31/551; A61K 31/4402; A61K 31/451; A61K 31/4045; A61K 31/343; A61K 9/08; A61K 31/5517

(52) U.S. Cl. CPC A61K 9/006; A61K 9/08; A61K 47/10; A61K 31/437; A61K 31/4985; A61K 31/519; A61K 31/5517; A61K 31/551; A61K 31/4402; A61K 31/451; A61K 31/4045; A61K 31/343; A61K 31/165

(57) ABSTRACT

A dosage form for oral administration, via the mucous membranes, of an active principle for accelerated sleep induction and/or for treating sleep disorders and/or for treating a central nervous system disorder, the active principle being lipophilic or amphiphilic and being in a dissolved state that is stable and complete in a hydro-alcoholic solution having 35% to 70% by weight of ethanol and 30% to 65% by weight of water, generating nanostructures of the active principle under consideration within the hydro-alcoholic solution, thereby enabling the active principle to cross the blood-brain barrier, the active principle being in base and/or salt form and belonging to the chemical families of sleep inducers or modulators of lipophilic or amphiphilic type and of molecular weight that is less than 1000 Da, the active principle being selected from the families of Imidazopyridines, including Zolpidem, the family of Cyclopyrrolones, including Eszopiclone, the family of Pyrazolopyrimidines, including Zaleplon, the family of Benzodiazepines, including Midazolam and Brotizolam, the family of H1-antihistamine sedatives, including Doxylamine and Cyproheptadine, and/or the family of Melatonin and Melatonin agonists, including Melatonin, Ramelteon, and Agomelatine, the volume of the hydro-alcoholic solution being less than or equal to 2 mL, and the active principle being present at a dosage that is less than or equal to 8 mg, all of the active principle being absorbed in transmucosal manner via the mucous membranes of the floor of the oral cavity, in particular via the gums/cheeks, via the gum area, via the cheeks, or under the tongue.

The present invention relates to a dosage form for administering an active principle for accelerated induction of sleep and/or for treating sleep disorders and/or for immediate emergency treatment of central nervous system disorders, such as an epileptic seizure.

In particular, the present invention relates to such a dosage form that is capable of guaranteeing immediate systemic administration via an oral mucous membrane of an active principle that is intended to act rapidly on the receptors of the central nervous system in order to promote psychomotor calming or rapid sleep of the subject to whom it is administered and/or to resynchronize an existing imbalance between waking and sleeping phases.

A first constraint is found to be that of needing to be effective in a short period of time, e.g. a few minutes after administering the active principle under consideration.

The invention also relates to its very specific compositions and to its various uses that make it possible to obtain rapid pharmacodynamic effectiveness by using very low effective dosages of the active principles.

Firstly, the following elements should be considered: sleep disorders, whether they stem from a physiological or psychological change in the patients that suffer from them, represent an increasing proportion of the world population. Insomnia is defined as the inability to initiate or to maintain sleep for periods of time that are recognized as being satisfactory for good health, namely in the range 6 hours (h) to 8 h of sleep per night. In most western countries, the consumption of hypnotics is increasing, although no study has proposed any genuinely convincing explanation for this phenomenon. In the rare studies that are available, half of the people seen in general practice suffer from insomnia, which is said to be mild in 15% to 17% of the cases, moderate for 12% à 17%, and severe for 19% à 23%, but many insomniacs never raise this question with their doctor. Independently of any possible comorbidities, the resulting disruptions to diurnal, physical, mental, and social functions spoil the quality of life of such subjects, in much the same way as for chronic illness. Epidemiology shows a positive statistical link between sleep disorders and psychiatric disorders (depression, anxiety, emotional problems, illegal-substance and alcohol abuse), and an increased risk of traffic accidents and work accidents, in particular as a result of the residual effects of the hypnotic molecules that are used. The number of Americans suffering from chronic sleep disorders has been estimated at at least 40 million, and 30% of the population experience this disorder according to studies in Europe and Australia. One French person in six complains about sleep, i.e. nearly 10 million people, and the prevalence of severe insomnia in the general population lies in the range 10% to 20%. It should be observed that, with age, sleep disorders become worse and more chronic, and 60% to 70% of consumers of hypnotics are more than 40 years old. In the USA, a recent study has indicated a prevalence of insomnia in one third of adults, with a total annual cost of insomnia lying in the range 92.5 to 107.5 billion dollars. In addition, insomnia leads to reduced effectiveness working: it seems that good sleepers spend twice as much time at work, studying, and communicating than insomniacs. Sleep disorders are well defined and explored by medical science, and in outline Health Authorities describe them as follows: “mild” insomnia relates to one night or less per week and presents minimum diurnal impact; “moderate” insomnia relates to two or three nights per week and presents diurnal impact of the type comprising fatigue, sullenness, tension, irritability; “severe” insomnia relates to four nights or more per week and presents diurnal impact of the type comprising fatigue, sullenness, tension, irritability, diffuse hypersensitivity, difficulty with concentration and degraded psychomotor performance.

In addition, circadian rhythm disorders are caused by day/night desynchronization, occurring most often in air passengers who travel over very long distances, passing rapidly across many time zones. The effects of time zone difference, commonly known as “jet lag”, manifest themselves as very disrupted sleep, known as the asymmetry phenomenon, that improves only gradually. Circadian rhythm disorders may also be caused by repeated night work.

Currently, there exist many pharmaceutical active principles for self-administering by the patient, either for inducing sleep more easily, or for resynchronizing the circadian rhythm. Such self-administration of the active principles that act on neural receptors or mechanisms of the central nervous system, generally takes place via the digestive tract. Unfortunately, the active principles are of lipophilic nature, and, like all active principles of this chemical nature, when they are introduced into the digestive tract and the stomach, they are subjected to the “digestive first-pass” effect, with degradations and losses associated with the medium in the stomach or with variations in intestinal physiologies. They are then subjected to the “hepatic first-pass” effect that causes them to be metabolized and/or to be degraded to a greater or lesser extent, with numerous metabolites being made, that are mostly inactive, but some of which are toxic.

Consequently, the quantity of active principle(s) that is actually bioavailable, and thus active in inducing sleep, may be extremely small, and examples are provided below to illustrate this: only a very residual fraction of the administered quantity thus remains available for producing the expected pharmacological effect.

It is also known that, on average, therapeutic effectiveness for the patient starts in the range 30 minutes (min) to 45 min after oral ingestion, corresponding to the time taken for digestive absorption, followed by metabolization, and finally by vascular diffusion towards the effector centers of the central nervous system, whether for inducing sleep or for resynchronizing wake/sleep balance.

As a result, two major problems can appear.

The first problem is that it is necessary to administer a dose to the patient that is sufficient, taking account both of the slow speed of digestive absorption, which is fragmentary, and also of the loss associated with hepatic metabolism, and then taking account of dilution in the biological liquids of the organism, in plasma, in blood cells, and in interstitial and intracellular liquids; such dispersion, in the organism, of dosages administered orally reduce correspondingly the useful molecular fraction of the active principle that can indeed reach its specific receptors in the central nervous system both rapidly and in a quantity that is pharacodynamically sufficient, whether for inducing sleep, or for resynchronizing the wake/sleep function.

The administration route and above all the administration method and the absorption and bioavailability yield of the administered active principle are thus determining factors for rapidly distributing a useful dose of active principle to the blood stream in the shortest possible time, and thus enabling it to act without delay on its effectors of the central nervous system.

Naturally, the person skilled in the art knows that injectable forms are forms of administration that are suitable for delivering rapid and determining action of the active principles: in particular, with regard to inducing sleep and to reducing vigilance, the anesthetist knows that intravenous administration is effective immediately, but this is vascular administration and requires highly qualified personnel and suitable monitoring.

It is clear that such intravenous administration cannot be recommended for use by hundreds of millions of subjects that wish to go to sleep quickly, or indeed for subjects with circadian rhythm disorder, who are in a situation that is likewise not suitable for such procedures. Furthermore, it is difficult to envisage a third person administering emergency treatment, e.g. of an epileptic seizure, in particular in a child, by the intravenous route.

In addition to the oral and intravenous routes, another route for administration is known that passes via the oral mucous membrane, which route enables medication to be administered by passively passing through the oral mucous membranes, whereby molecules, if they are sufficiently absorbed by the mucous membranes, can pass into the veins under the tongue and in the cheeks, into the right side of the heart, into the pulmonary artery, and then reach the left side of the heart so as to leave via the aorta and be distributed to the general arterial blood stream, thereby short-circuiting the digestive tract and the hepatic metabolism to which medication that is administered orally is subjected.

Nevertheless, existing formulations that are administered via the oral mucous membranes are not satisfactory and are used very little for treating sleep anomalies, in particular as a result of the active principles being made up of molecules that, by their nature, are not very soluble or are insoluble in biological liquids such as saliva. Since those lipophilic molecules are not able to dissolve on contact with saliva, they generally remain in crystalline and/or aggregate form, thus making them impossible to absorb via the mucous membranes and thus making it impossible for them to pass into the general blood stream; they are thus mainly swallowed and are subjected to the above-described effects relating to the oral administration of medication.

The prior art has already made attempts to administer lipophilic active principles having hypnotic activity via the oral mucous membrane, and some of those attempts may be found in documents WO 2008/141264, WO 2006/089082, US 2007/0248548, WO 2007/123955, US 2008/013929, CA 2 582 007, and US 2004/265239. Those documents differ from the invention in particular by administering in spray form. This requires the use of specific ingredients. Such spray forms also present other drawbacks. In particular, inaccurate spraying into the oral cavity produces airborne diffusion and a dispersion that inevitably causes a constant loss of a sizable fraction of the administered dose, which mixes immediately with saliva and is swallowed. Another difference relates to the use, by the invention, of a high ethanol content (at least 35% by weight), which is not envisaged in the prior-art documents. Those documents are thus incapable of providing the technical advantages obtained by this high ethanol content. Other differences also exist.

An object of the present invention is to provide a dosage form that does not have the above-mentioned drawbacks.

Another object of the present invention is to provide a dosage form that is simplified and that is very rapidly bioavailable in the arterial blood stream, given that the brain takes the greatest flow rate of arterial blood in the organism, taking 2 liters (L) of blood per minute (/min). The invention also seeks to provide a particular method of administration by depositing the solution of the invention completely in the groove between the gum and the cheek, which method is accessible to the average consumer and that makes it possible to administer an accurate quantity of an active principle, becoming immediately bioavailable in the blood stream, so as to be able to treat, very rapidly and effectively, particular syndromes such as those of sleep difficulties, sleep-instability disorders, circadian rhythm disorders, or epileptic seizures, with low useful doses of active principle that are available immediately in the blood stream, and that are immediately pharmacologically active on the receptors of the central nervous system. This provides the same advantages as an intravenous administration, but naturally without the technical requirements nor the risks of infection, nor the costs of equipment and of specialized medical personnel trained to use it.

The present invention thus provides a dosage form for oral administration, via the mucous membranes, of an active principle for accelerated sleep induction and/or for treating sleep disorders and/or for treating a central nervous system disorder, said active principle being lipophilic or amphiphilic and being in a dissolved state that is stable and complete in a hydro-alcoholic solution comprising 35% to 70% by weight of ethanol and 30% to 65% by weight of water, generating nanostructures of said active principle under consideration within the hydro-alcoholic solution, thereby enabling the active principle to cross the blood-brain barrier, said active principle being in base and/or salt form and belonging to the chemical families of sleep inducers or modulators of lipophilic or amphiphilic type and of molecular weight that is less than 1000 daltons (Da), said active principle being selected from the families of Imidazopyridines, including Zolpidem, the family of

Cyclopyrrolones, including Eszopiclone, the family of Pyrazolopyrimidines, including Zaleplon, the family of Benzodiazepines, including Midazolam and Brotizolam, the family of H1-antihistamine sedatives, including Doxylamine and Cyproheptadine, and/or the family of Melatonin and Melatonin agonists, including Melatonin, Ramelteon, and Agomelatine, the volume of said hydro-alcoholic solution being less than or equal to 2 milliliters (mL), and said active principle being present at a dosage that is less than or equal to 8 milligrams (mg), all of said active principle being absorbed in transmucosal manner via the mucous membranes of the floor of the oral cavity, in particular via the gums/cheeks, via the gum area, via the cheeks, or under the tongue.

Advantageously, the hydro-alcoholic solution comprises 40% to 65% by weight of ethanol and 35% to 60% by weight of water.

Advantageously, the hydro-alcoholic solution contains a pH adjusting agent.

Advantageously, said active principle contains a carbonyl-acid function, said hydro-alcoholic solution containing a pH adjusting agent and/or a sequestering agent.

Preferably, the hydro-alcoholic solution is made up exclusively of ethanol, water, and an active principle. Specifically, dissolving the active principle in a hydro-alcoholic solution makes it possible to create nanostructures, having an average size that is about 1 nanometer (nm), which nanostructures are particularly concentrated in an equilibrium that occurs as from 35° ethanol, the optimum concentrations of said nanostructures being found to be optimum in high degrees of ethanol, in the range 45° to 65°, of the hydro-alcoholic solutions of the invention. It turns out that some active-principle nanostructures have demonstrated a specific affinity and a capacity to cross the blood-brain barrier that is at least ten time greater than that of an intravenous solution of the same active principle.

Advantageously, all of said active principle is absorbed transmucosally in less than 10 seconds (s), advantageously in less than 6 s.

Advantageously, the molecular weight of said active principle is less than 600 Da.

Advantageously, the unit dose of said active principle is less than 5 mg.

Advantageously, the volume of hydro-alcoholic solution is less than or equal to 1 mL. Advantageously, said active principle does not require any dissolving or solubilizing or stabilizing additive within said hydro-alcoholic solution. Specifically, in the event of the active principle being unstable within said hydro-alcoholic solution, the solution may be prepared extemporaneously prior to being administered, e.g. by means of devices known for this purpose, enabling the active principle to be properly dissolved immediately, and then to be administered, such as with the devices described in documents FR 2 939 321 and FR 2 930 140.

The term “via the membranes” means any passive passage of a lipophilic or amphiphilic molecule that is presented in its dissolved state, and this enables it, as a result of its intrinsic lipophilicity, to be absorbed spontaneously through the mucous membranes that are themselves lipophilic, in particular via the cheeks, via the gums, via the gum area, or under the tongue, all making up the floor of the oral cavity, which serves both as a physical support and as a kind of container. Furthermore, these particular mucous membranes make it possible to concentrate the liquid deposit on a preferred absorption surface that is particularly suitable for best operation of this application.

The term “dissolved state that is stable and complete of the active principle” means a dissolved state that restores an active principle to its molecular state in the medium in which it is dissolved, with the dissolved state being obtained in a water and ethanol solution with a high degree of ethanol, and this enables nanostructures of the active principles to be constituted around an average size of 1 nm, which are preferably absorbed through the blood-brain barrier, said dissolved state also preventing any possibility of unwanted recrystallization.

The term “hydro-alcoholic solution at X degrees of alcohol” means a solution that presents a degree X of alcohol, corresponding to the ratio of the volume of pure alcohol)(100° contained in the hydro-alcoholic solution to the total volume of the solution. The degree of alcohol of the hydro-alcoholic solution varies as a function firstly of the degree of alcohol used to form the solution, and secondly of the water/alcohol ratio of the solution. For example, for an initial alcohol at 100 degrees and a water/alcohol ratio of 50/50, the hydro-alcoholic solution is at 50 degrees of alcohol.

In the meaning of the present invention, the term “active principle acting on the central nervous system, on inducing sleep and/or on resynchronizing wake/sleep functions” is used to designate any lipophilic or amphiphilic active principle that is capable of acting via the arteries directly on the central nervous system on mechanisms that reestablish psychomotor equilibriums and/or that induce sleep and/or that rebalance wake/sleep synchronization, with this happening while being delivered without delay to the systemic blood stream, while using doses that are smaller and more effective than the unit doses usually administered orally. Specifically, the flow of blood to the brain conveyed by the two carotid arteries is the greatest flow in the organism, being of the order of 2 L/min. This cerebral arterial route thus represents a preferred direct access for a molecule acting on sleep or psychomotor-equilibrium parameters.

The active principle acting on the central nervous system and/or acting on inducing sleep and/or acting on resynchronizing wake/sleep functions, is present in base form and/or in salt form, and, by way of non-limiting example, in the form of Succinate, Chlorhydrate, Sulfate, Acetate, Tartrate, Citrate, Methylsulfate, or Borate, or of any pharmaceutically compatible salts.

The active principle of the invention is selected from any lipophilic or amphiphilic active principle of low molecular weight, typically less than or equal to 1000 Da, that has sleep inducing activity or activity of rebalancing the functioning of the central nervous system and/or of rebalancing sleep, without any undesirable effects appearing on waking. By way of non-limiting example, mention may be made of molecules that are described and formulated in particular applications of the invention: hypnotics forming part of the family of Benzodiazepines, which hypnotics are anxiolytic and hypnotic molecules that form part of the class of psychotropic drugs, i.e. substances that modify the psyche and human behavior by acting on the central nervous system; or molecules similar to Benzodiazepines, such as Imidazopyridines, Cyclopyrrolones, or Pyrazolopyrimidines, for example; or H1-antihistamines of sedative type, such as Doxylamine; or molecules such as Melatonin and analogs of Melatonin, which molecules are both active in inducing sleep and in resynchronizing circadian rhythms.

The mode of action of Benzodiazepines and the like, is the potentiation of the inhibitory effect of Gamma AminoButyric Acid (GABA) in the central nervous system The pharmacological name of each Benzodiazepine usually ends with the suffix “azopam” or “azolam” or “azepam”; by way of non-limiting example, mention may be made of Flunitrazopam (known under the trade name Rohypnol®), Lormetazepam (Noctamide®), Lorazepam (Temesta®), Bromazepam (Lexomil®), Clotiazepam (Veratran®), Oxazepam (Seresta®), Temazepam (Normison®), Loprazolam (Havlane®). “Similar” molecules are included in this family of Benzodiazepines, such as Cyclopyrrolones, including Eszopiclones, or Imidazopyridines, including Zolpidem (Stilnox®). It should be observed that these substances are pharmacologically potency, since they are administered orally at low unit doses, e.g. in the range 0.5 mg to 5 mg.

The active principles suitable for being formulated in accordance with the invention are lipophilic or amphiphilic molecules that are made soluble in special hydro-alcoholic solutions, for each of said active principles acting on a central nervous system disorder and/or on inducing sleep or on adjusting wake/sleep balance. The active principles are preferably of lipophilic or amphiphilic nature and are of low molecular weight, typically less than 1000 Da.

The dosage form of the invention is in the form of a hydro-alcoholic solution lying in the range 35% to 70% by weight of alcohol, and in the range 30% to 65% by weight of water. Advantageously, the hydro-alcoholic solution lies in the range 40% to 65% by weight of alcohol, and in the range 35% to 60% by weight of water. The active principle(s) thus pass(es) into the systemic blood stream in a hydro-alcoholic solution that has a degree of alcohol that varies, preferably lying in the range 35° to 70° and still more preferably in the range 40° to 65°. Thus, by the constitution of specific nanostructures of the active principle under consideration, which nanostructures are due to the particular equilibrium of dissolution produced between the high degree of ethanol and water, the only elements involved in dissolution, the invention increases, in unexpected and scientifically proven manner, the capacity of the active principle under consideration to cross the blood-brain barrier. Constituting the nanostructures requires a high degree of ethanol, at least equal to 35°, and the absence of any additional dissolution or stabilization ingredient, and the absence of any additive ingredient that might apply to the organoleptic characteristics of the composition. The alcohol used in the present invention is preferably ethanol.

According to an advantageous characteristic of the invention, alcohol does not only act as a solvent and a constituent, together with the water, of said active-principle nanostructures, but it also acts to promote accelerated absorption via the mucous membrane, at a speed that increases as a function of increasing degree of alcohol used. Specifically, at a high degree, the small ethanol molecule dissolves the surface layer of the oral mucous membrane, which surface layer is made up of lipidic structures. It thus makes it easier for the dissolved lipophilic molecules to access the outer membrane of the epithelium of the oral mucous membrane, which outer membrane is itself lipophilic. Furthermore, since ethanol has a low molecular weight (MW=46 Da), it thus represents a strong osmotic vector for amplifying rapid absorption via the mucous membrane of said molecules in solution.

In a preferred embodiment of the invention, the hydro-alcoholic solution is based on water and ethanol. Advantageously, it comprises only water, ethanol, and an active principle.

Any addition of excipient(s) may weaken the forming of the nanostructures of the active principle under consideration, and may also weaken the stability over time of the hydro-alcoholic solution, and may even drastically reduce the absorbed dose ratio, while simultaneously reducing the speed of absorption of the active principle under consideration via the mucous membrane. It should be observed that although known for being very disagreeable, the taste of some active principles, when deposited in stable and complete hydro-alcoholic dissolution in accordance with the invention via the cheeks, via the gums, via the gum area, or under the tongue, is not noticed by patients. Specifically, these particular mucous membranes, in particular the gum/cheek mucous membranes, do not have taste receptors, and small-volume hydro-alcoholic solutions of the invention are absorbed via these mucous membranes in a time of a few seconds as a result of their high degree of alcohol. These molecules thus never reach the taste receptors in the mouth, which receptors are situated in the papillae on the top surface of the tongue, or in the upper portion of the rear of the mouth known as the cavum. The invention thus makes it possible to avoid possible taste problems that are common to many medications by selectively administering the composition of the invention in contact with certain mucous membranes that, physiologically, do not have taste receptors.

In a particular embodiment, in particular when the active principles to be administered contain a carbonyl-acid function, the dosage form of the invention may also contain a pH adjusting agent and/or a sequestering agent. Specifically, the active principles containing a carbonyl-acid function may react with primary alcohols and secondary alcohols so as to form an ester. This reaction leads to reduction in the active principle content and to the appearance of impurities, which is incompatible with preparing a medication. The addition of at least one pH adjusting agent makes it possible to modulate the proportion of lipophilic and hydrophilic-ionized base forms of the active principle, so as to optimize the bioavailability via the mucous membrane of each active principle, in order to absorb the administered dose in the most rapid and complete manner. Preferably, the pH adjusting agent is selected from sodium carbonates and bicarbonates, monosodium or disodium phosphates, triethanolamine, sodium hydroxide (NaOH), and potassium hydroxide (KOH). The sequestering agent is preferably selected from ethylenediaminetetraacetic acid (EDTA), calcium disodium ethylenediaminetetraacetate (E385), glucono delta lactone (E575), sodium gluconate (E576), potassium gluconate (E577), and sodium tripolyphosphate, or from acid pH adjusting agents such as hydrochloric acid, tartaric acid, lactic or citric acids, or any pharmaceutically acceptable salts thereof.

The hydro-alcoholic solution may be pre-established in stable manner or it may be made up extemporaneously at the moment of use, e.g. by means of a device that is suitable for preparing it and for administering it, such as the devices described in documents FR 2 939 321 and FR 2 930 140.

The dosage form of the invention enables the active principle to pass passively via the lipophilic oral mucous membranes, mainly via the cheeks, via the gums, via the gum area, or under the tongue, in a time of less than 10 s after depositing the hydro-alcoholic solution in contact therewith. This very rapid absorption time thus makes it possible to prevent the solution and the active principle(s) from stagnating in any way in the oral atmosphere, and to prevent them from mixing in unwanted manner with saliva, which might spoil either the active principle itself, or the specific equilibrium of its complete and stable dissolution, which would introduce a break in the continuity and stability of the dissolution of the active principle, and would have a negative impact on establishing the nanostructures of said active principle. The short absorption time also makes it possible to prevent any of the administered hydro-alcoholic solution and the active principle that it contains from being swallowed in reflex manner.

Since the outer epithelial membrane of the oral mucous membrane is made up of phospholipidic structures that absorb the lipophilic or amphiphilic molecules passively, and by elective affinity, the passage of such an active principle, presented in the dissolved state of the invention, through the mucous membranes is based on an osmotic attraction towards the other side of said membrane, which attraction depends both on the concentration of dissolved active principle and on the concentration of alcoholic solution under consideration. The osmotic attraction is stronger and more potent when the lipophilic molecule in the dissolved state is of low molecular weight and when the degree of alcohol that serves to promote absorption is high.

The lipophilic oral mucous membranes, mainly of the cheek, the gum, the gum area, and under the tongue, possess a network of micro-vessels that is very dense and that is almost spongy, so much so that the molecules, both the alcohol solvent and the dissolved active principle, that pass through the lipophilic pores of the epithelial membrane are immediately captured by the microcirculation of blood and are directed towards the veins under the tongue. This phenomenon is accentuated by the presence of alcohol that causes vasodilatation and an increase in the local microvascular flow of the mucous membranes. As a result of this raised blood flow, increased locally by the alcohol, there is thus never any equilibrium across the membrane: the concentration in the mouth continues to remain greater, until the mechanism is depleted as a result of there being no more molecules to be absorbed.

Thus, all of the alcohol and all of the active principle that has been dissolved passes through the mucous membrane in a time of 4 s to 10 s, on average, for a volume of 1 mL at 50° of ethanol, as has been demonstrated by significant pharmacoclinical studies.

Using the dosage form of the invention makes it possible to administer a dose of active principle that is thus immediately absorbed as soon as it is deposited in contact with the pre-selected mucous membrane, so as to be distributed instantly via the central vascular system to the arterial zones of the organism, thus being able to act pharmacologically without delay and being exempt from the major effects of the digestive and hepatic passes known for oral administration.

The hydro-alcoholic solution, with an alcohol content of at least 35% by weight, also presents the advantage of solubilizing active principles even if they are not very soluble, and of protecting the pharmaceutical formulation from microbiological contamination, without having to add antimicrobial-preservation agent(s) thereto of the Paraben type, that are known for raising considerable reservations in terms of health safety.

As described below by particular examples and dosages by weight of active principle(s), the dosage form of the invention enables active pharmacological substances to be administered in low and useful doses both systemically and immediately in order to treat a disorder in the central nervous system and/or for rapidly inducing sleep and/or for resynchronizing wake/sleep physiological functions.

In particular, the dosage form of the invention may be used for making a medication for treating and/or preventing sleep disorders, and it presents numerous advantages compared with an oral or injectable form, and in particular great simplicity of use, and an almost-immediate distribution to the central nervous system of a useful low dose of active principle, making it possible to induce the desired pharmacological effect without delay or loss to the remainder of the organism. Specifically, the arterial flow of blood to the brain is the greatest flow in the organism and the active principle administered by the invention thus reaches its pharmacological target directly, firstly via the arteries, before being distributed and disseminated into the various compartments of the organism. The invention provides a pharmacodynamic result in a time that is more accessible than the time typically required with intravenous use, while administering an effective dosage that is substantially less than the dosage required for oral administration.

Advantageously, the present invention offers great simplicity and very good dosage-form stability: adjusting the equilibrium of the water/alcohol solution, specifically established for each molecule, guarantees that the active principle can be solubilized in a small hydro-alcoholic volume, less than or equal to 2 mL of solution, and preferably less than or equal to 1 mL. Furthermore, the invention eliminates most excipients that are essential for usual oral pharmaceutical forms and for traditional under-the-tongue forms, or for spray forms. It thus makes it possible both to reduce manufacturing costs, and to reduce the risks of intolerance and the possible interactions between active principle(s) and excipients.

Furthermore, since the active principle does not encounter any significant obstacle to its assimilation and its immediate distribution in the organism, the basic dose administered may be very low, closer to the useful dose for fulfilling the required pharmacological activity. The dose is preferably less than or equal to 8 mg, and advantageously less than 5 mg.

Singularly, the time to act is very short, in particular compared with the slow speed at which medication is absorbed via the digestive tract. Almost-immediate pharamacological delivery enables a patient to administer a substance for an effect that is almost equivalent to the effectiveness of a flash intravenous injection into the blood stream.

In addition, lipophilic oral mucous membranes, mainly in the cheeks, the gums, the gum area, and under the tongue, have a total absorption surface area that is increased by their naturally wrinkled epithelial tissue, so administering the dosage form of the invention is without any risk of accidental swallowing or taking the wrong route. Specifically, it enables extremely rapid passage through the mucous membranes that prevents the administered active principles from being diluted by saliva or swallowed, with the advantage of not destabilizing the mucous membranes with wetting-agent derivatives, e.g. as occurs with many formulations that pre-exist the invention.

In addition, the effects of the alcohol are insignificant. By way of example, the dose of alcohol administered in 1 mL of hydro-alcoholic solution at 50° of ethanol would, if it were ingested, represent a maximum dose distributed via the systemic blood stream of 0.00785 grams per liter (g/L) of ethanol, i.e. only about one sixtieth of the blood-alcohol limit of 0.5 g/L set by legislation in France. In fact, in the context of administration via the mucous membrane of the invention, the ethanol that has served as a vector for the active principle is breathed out for the most part in the volatile state while passing through the pulmonary alveoli to which it is delivered in a few seconds via the pulmonary artery, from the right ventricle. Since this alcohol has not been ingested, after its initial pass through the lungs and being breathed out, there is thus practically no longer any ethanol distributed to the organism, which constitutes a notable advantage of the invention. Specifically, ethanol is a known vector that does not remain in the organism after enabling the active principle(s) administered at effective low dosages to pass rapidly into the systemic blood flow. The invention thus enables the active principle(s) both to be administered and to have rapid pharmacodynamic action, while significantly reducing the intolerance phenomena that may be associated with high dosages of active principles and/or with excipients and stabilizers of such active principles. By using a vector such as ethanol that is evacuated from the organism very quickly via the respiratory route before exerting any effect on the organism, and by not requiring excipients, the invention reduces risks and costs compared with oral formulations and complex formulations, such as the formulations mentioned above, for administration via the mucous membrane, in documents US 2007/0248548, WO 2007/123955, US 2008/013929, CA 2 582 007, and US 2004/265239.

Preferably, the dosage form of the invention is associated with specific industrial packaging in order to prevent the active principle(s) from degrading on contact with the air. By way of example, a particular embodiment consists in using opaque flexible unitary packaging that is provided with an administration cannula having a length that may be as long as several centimeters, thereby making it possible to deposit the entire hydro-alcoholic volume as a spot, administered in contact with a zone of the mucous membrane that has been determined precisely for this purpose. The unitary packaging may also be filled in a nitrogen atmosphere so as to better protect the stability of the composition, and it may be impermeable to oxygen and to light. The packaging guarantees that the active principles that are dissolved in hydro-alcoholic solution of the invention are stable over time. For comfort in use by the patient and/or for easy transport, packages of the “stick” type, in the form of specific leaktight vials, may preferably be used. Still more preferably, the dosage form of the invention is packaged in single-dose sticks of 0.1 mL to 2 mL, suitable for providing a dose of active principle that is enough for a single administration. Advantageously, this packaging is easy to transport, and enables the dosage form to be used easily, discreetly, and rapidly. Said “stick” may also present particular characteristics that, by way of example, make it possible to mix the active principle and hydro-alcoholic solution immediately in extemporaneous manner, and to enable it to be administered immediately, in accurate manner, in particular by means of a suitable cannula, as described in documents WO 2010/063978 and WO 2009/016309, for example. Other characteristics and advantages appear from the following examples of the invention.

The examples provided by the invention all have the same common constraints, namely a lipophilic or amphiphilic active principle that is soluble in a mixture of alcohol and water.

Said active principle, which is of low molecular weight, typically less than 1000 Da, advantageously less than 600 Da, may be used in base and/or salt form, the base being preferred wherever possible. For best effectiveness, the active principles should have a period of action that is limited to a few hours so as not to generate side effects, in particular after inducing sleep in insomniacs, so as not to keep said same insomniacs in a situation of residual drowsiness which could interfere with, and adversely affect, the conditions of their active life once they are awake.

Another common characteristic of the molecules is their degradation, to a greater or lesser extent, by the hepatic metabolism when they are administered orally. Specifically, the invention enables the useful dose of active principle under consideration to be immediately bioavailable in nerve centers and as a result enables the useful dosage to be significantly reduced, thereby resulting in a triple saving of action time, of administered dose, and consequently of side effects that are mainly associated with the metabolites that are inevitably created by the hepatic activity receiving the same dosages orally. Thus, for inducing sleep, active principles having what is known as a “half-life” in the organism that is substantially longer than the recommended duration for sleep, i.e. 7 h to 9 h, are not preferred applications of the invention, even if the active principles could be used in the form of the invention.

### EXAMPLES

I—Hypnotic and Inducive Molecules for Inducing Sleep and/or for Treating a Central Nervous System Disorder:

### 1—Family of Imidazopyridines:

Zolpidem is one of the major hypnotics on the market. This molecule, forming part of the family of Imidazopyridines, is a molecule of low molecular weight (MW=307.4 Da) that is very lipophilic and, as a result, is 92% bonded to plasma proteins in the blood stream. It presents the advantage of having a Zolpidem base form that is quite soluble in ethanol, which is favorable. Like many lipophilic substances, its bioavailability when taken orally is not greater than 70% of the ingested dose. Zolpidem is usually dosed at 5 mg or 10 mg per tablet. Zolpidem presents a short half-life of 2 h to 3 h in the organism, and this makes it an application that corresponds to the criteria of the invention. A formulation of the invention thus takes into account that a useful unit dose administered via the oral mucous membranes should not be greater than 8 mg, as a result of the rapid speed at which the formulation of the invention is distributed in the blood compared with an oral form or a spray form. As a result, the invention makes it possible to use low-dosage formulations that take account of the “bolus” effect associated with the rapid systemic vascular passage of the Zolpidem active principle, while being administered via the oral mucous membrane.

In the context of the present invention, the dosage of Zolpidem advantageously lies in the range 0.025 mg to 7 mg, preferably in the range 0.2 mg to 6 mg, and more preferably in the range 0.5 mg to 5 mg.

It should be observed that for oral forms, it is a Zolpidem salt, namely Zolpidem tartrate, that is often used, in particular as described in document US 2004/265239 which describes a treatment for insomnia referred to as NovaDel's Zolpimist™ (Zolpidem tartrate) 5 mg and 10 mg Oral Spray. The Zolpidem tartrate described in that document is less soluble in water and in ethanol than Zolpidem base, since the Zolpidem base dissolves in ethanol at 50 mg/mL, which makes the application of the invention easier. Nevertheless, Zolpidem tartrate could also be used in the context of the present invention.

### Formulation A1: 1 mL for 5 mg of Zolpidem Base:

distilled water: 0.45 mL

ethanol: 0.55 mL

Zolpidem base: 5 mg

### Formulation B1: 0.75 mL for 3 mg of Zolpidem Base:

distilled water: 0.3375 mL

ethanol: 0.4125 mL

Zolpidem base: 3 mg

### Formulation C1: 0.5 mL for 2 mg of Zolpidem Base:

distilled water: 0.25 mL

ethanol: 0.25 mL

Zolpidem base: 2 mg

### Formulation D1: 0.3 mL for 1.5 mg of Zolpidem Tartrate:

distilled water: 0.155 mL

ethanol: 0.145 mL

Zolpidem tartrate: 1.5 mg

NaOH q.s. pH 6 to 8

### Formulation E1: 0.3 mL for 1 mg of Zolpidem Base:

distilled water: 0.152 mL

ethanol: 0.148 mL

Zolpidem base: 1 mg

### Formulation F1: 0.25 mL for 0.5 mg of Zolpidem Base:

distilled water: 0.1245 mL

ethanol: 0.1255 mL

Zolpidem base: 0.5 mg

In order to guarantee both accurate deposition and rapid and complete absorption via the mucous member of the Zolpidem active principle, such administration is preferably performed in practical manner in the anatomical groove between the gum and the cheek, constituted on the outside by the mucous membrane of the cheek, and on the inside, by the strip of mucous membrane on the gum. Thus, this anatomical quasi-container that is the groove between the gum and the cheek, protects the hydro-alcoholic solution that is deposited therein from being mixed with saliva and/or swallowed, and enables it to be completely absorbed towards the vascular system in a time of 4 s to 6 s for a volume of solution that is preferably 1 mL or less.

Administered in the conditions of the invention, the calming effect produced by Zolpidem appears in the brain within only a few minutes for the dosages of the invention as given in the examples, since the arterial blood flow in the brain is the greatest arterial blood flow of the organism, whereas, when administered orally, the effect of Zolpidem is perceptible only after at least 15 min and after absorbing doses of 5 mg or 10 mg.

### 2—Family of Cyclopyrrolones:

Eszopiclone, which forms part of the family of Cyclopyrrolones, presents an average half-life of 6 h. It is a dextrorotatory isomer of Zopiclone, a molecule that is not very soluble in water and alcohol. Eszopiclone is a lipophilic molecule of low molecular weight (MW=388 Da), 60% of it being bonded to plasma proteins, that is generally administered orally at a dosage of 1 mg, 2 mg, or 3 mg.

In the context of the present invention, the dosage of Eszopiclone advantageously lies in the range 0.025 mg to 2 mg, preferably in the range 0.2 mg to 2.5 mg, and more preferably in the range 0.15 mg to 1 mg.

### Formulation A2: 1 mL for 1 mg of Eszopiclone Base:

distilled water: 0.45 mL

ethanol: 0.55 mL

Eszopiclone base: 1 mg

Hydrogen phosphate pH adjuster of Na+phosphoric acid q.s. pH 4 to 6

### Formulation B2: 0.5 mL for 0.8 mg of Eszopiclone Base:

distilled water: 0.24 mL

ethanol: 0.26 mL

Eszopiclone base: 0.8 mg

Hydrogen phosphate pH adjuster of Na+phosphoric acid q.s. pH 4 to 6

### Formulation C2: 0.5 mL for 0.5 mg of Eszopiclone Base:

distilled water: 0.25 mL

ethanol: 0.25 mL

Eszopiclone base: 0.5 mg

Hydrogen phosphate pH adjuster of Na+phosphoric acid q.s. pH 4 to 6

### Formulation D2: 0.3 mL for 0.25 mg of Eszopiclone Base:

distilled water: 0.15 mL

ethanol: 0.15 mL

Eszopiclone base: 0.25 mg

Hydrogen phosphate pH adjuster of Na+phosphoric acid q.s. pH 4 to 6

### Formulation E2: 0.3 mL for 0.15 mg of Eszopiclone Base:

distilled water: 0.15 mL

ethanol: 0.15 mL

Eszopiclone base: 0.15 mg

Hydrogen phosphate pH adjuster of Na+phosphoric acid q.s. pH 4 to 6

### 3—Family of Pyrazolopyrimidines:

Zaleplon, which forms part of the family of Pyrazolopyrimidines, is a molecule of low molecular weight (MW=305.34 Da), lipophilic with a bioavailability reduced to only 30% of the dose administered orally. It presents a short half-life of only 1 h in the organism, and this constitutes an application that is particularly suited to the invention, so as to generate sleep rapidly by administering very low doses that are distributed immediately after being absorbed via the oral mucous membrane. When administered orally, it is generally administered at unit dose of 5 mg or 10 mg. In view of its low bioavailability, it should be understood that the invention may make it possible to use lower doses that are rapidly active compared with the usual doses taken orally.

In the context of the present invention, the dosage of Zaleplon advantageously lies in the range 0.025 mg to 5 mg, preferably in the range 0.2 mg to 4 mg, and more preferably in the range 0.5 mg to 3 mg.

### Formulation A3: 1 mL for 3 mg of Zaleplon Base:

distilled water: 0.50 mL

ethanol: 0.50 mL

Zaleplon base: 3 mg

### Formulation B3: 0.75 mL for 1.5 mg of Zaleplon Base:

distilled water: 0.355 mL

ethanol: 0.395 mL

Zaleplon base: 1.5 mg

### Formulation C3: 0.5 mL for 1 mg of Zaleplon Base:

distilled water: 0.235 mL

ethanol: 0.265 mL

Zaleplon base: 1 mg

### Formulation D3: 0.3 mL for 0.5 mg of Zaleplon Base:

distilled water: 0.15 mL

ethanol: 0.15 mL

Zaleplon base: 0.5 mg

### 4—Family of Benzodiazepines:

### A. Midazolam:

This molecule, forming part of the family of Benzodiazepines, is generally taken orally at doses of 7.5 mg or 15 mg, or 2 mg/mL in drinkable form, or in injectable form at 1 mg/mL or 5 mg/mL, made water-soluble by using its hydrochloride salt. It has a low oral bioavailability, only 36% of the administered dose, and it bonds strongly to plasma proteins. Specifically, it is a lipophilic molecule that is not soluble in water (solubility of only 0.024 mg/mL) and is of low molecular weight (MW=321 Da), but it is freely soluble in ethanol, thereby making it good for application in the context of the present invention.

In the context of the present invention, the dosage of Midazolam advantageously lies in the range 0.15 mg to 5 mg, preferably in the range 0.3 mg to 4 mg, and more preferably in the range 0.5 mg to 3 mg.

### Formulation A4a: 1 mL for 3 mg of Midazolam Base:

distilled water: 0.50 mL

ethanol: 0.50 mL

Midazolam base: 3 mg

### Formulation B4a: 0.75 mL for 2 mg of Midazolam Base:

distilled water: 0.375 mL

ethanol: 0.375 mL

Midazolam base: 2 mg

### Formulation C4a: 0.5 mL for 1.5 mg of Midazolam Base:

distilled water: 0.25 mL

ethanol: 0.25 mL

Midazolam base: 1.5 mg

### Formulation D4a: 0.5 mL for 1 mg of Midazolam Chlorhydrate:

distilled water: 0.25 mL

ethanol: 0.25 mL

Midazolam chlorhydrate: 1 mg

pH buffer q.s. pH 6 to 8

### Formulation E4a: 0.3 mL for 0.5 mg of Midazolam Base:

distilled water: 0.15 mL

ethanol: 0.15 mL

Midazolam base: 0.5 mg

In the invention, a particular application of Midazolam, which is an emergency therapy, is the immediate treatment of epileptic seizures, in particular in children.

In this event, the application of the invention makes it possible, by moving the patient's cheek outward, to deliver a small volume of hydro-alcoholic solution having a high level of ethanol into contact with the gum and cheek, thereby making it possible to deliver the active principle to the cerebral blood stream in a time of a few seconds only, and to sedate the seizure.

For this treatment, it is particularly important to have the specific characteristics of the invention, which characteristics make it possible to deliver the effective dosage of Midazolam via the gum and cheek, the small volume of the invention preventing any administration by a wrong route, and the high degree of ethanol guaranteeing passage via the mucous membranes that is much more rapid compared with available treatments.

By way of example, a formulation specifically adapted for treating epileptic seizures may correspond in terms of dosage and volume, and in non-limiting manner as a function of the weight and age of the subject under consideration, to the following formulations:

### Formulation F4a: 0.75 mL for 5 mg of Midazolam Base:

distilled water: 0.30 mL

ethanol: 0.45 mL

Midazolam base: 5 mg

### Formulation F4b: 0.4 mL for 3.5 mg of Midazolam Base:

distilled water: 0.16 mL

ethanol: 0.24 mL

Midazolam base: 3.5 mg

### Formulation F4c: 0.25 mL for 2 mg of Midazolam Base:

distilled water: 0.09 mL

ethanol: 0.16 mL

Midazolam base: 2 mg

### B. Brotizolam:

This is a lipophilic molecule of average molecular weight (MW=393.7 Da), with oral bioavailability that varies, being around 50% of the administered dose. It presents the advantages of having a short half-life of 4.4 h on average, and of being effective at low unit doses. Specifically, as a hypnotic sleep inducer, its oral dosage is extremely low, namely in the range 0.125 mg to 0.250 mg. Its relative solubility in ethanol enables it to be applied to the invention. In such conditions, the useful dose for inducing sleep is reduced by means of the invention, which guarantees its immediate vascular distribution.

In the context of the present invention, the dosage of Brotizolam advantageously lies in the range 0.015 mg to 0.3 mg, preferably in the range 0.02 mg to 0.180 mg, and more preferably in the range 0.025 mg to 0.1 mg.

### Formulation A4b: 1 mL for 0.1 mg of Brotizolam Base:

distilled water: 0.45 mL

ethanol: 0.55 mL

Brotizolam base: 0.1 mg

### Formulation B4b: 0.75 mL for 0.075 mg of Brotizolam Base:

distilled water: 0.35 mL

ethanol: 0.40 mL

Brotizolam base: 0.075 mg

### Formulation C4b: 0.5 mL for 0.05 mg of Brotizolam Base:

distilled water: 0.225 mL

ethanol: 0.275 mL

Brotizolam base: 0.05 mg

### Formulation D4b: 0.3 mL for 0.025 mg of Brotizolam Base:

distilled water: 0.135 mL

ethanol: 0.165 mL

Brotizolam base: 0.025 mg

It should be remembered that treatments for inducing sleep are considered as iterative, i.e. Health Authorities state that they should not be used for more than a few days, and they are usually used for treating short-term insomnia.

### II—Other Sleep Inducers, H1-Antihistamine Sedatives:

First generation “H1” antiallergic antihistamines are medications for the purpose of improving the comfort of allergic subjects. When they are administered orally, some of them also cause a sleep inducing sedative effect. The invention makes it possible to potentialize this sedative effect in order to make it occur at a useful dosage that is low, and in a time of only 5 min to 10 min, and consequently particularly low compared with the useful dosage obtained orally. Examples are given in application of the invention relating to Doxylamine, and relating to another antihistamine, Cyproheptadine that acts as a sedative.

### 5—Doxylamine:

Doxylamine, an H1-Antihistamine, is a small amphiphilic molecule (MW=270.37 Da for the Base, and 388.5 Da for the Succinate) with a bioavailability of only 24.7% of the ingested dose. Taken orally as an inducer of sleep at a unit dose of 15 mg, its peak plasma levels occur late, around 1 h to 2 h after ingestion, with a half-life around 3.5 h. The recommended dosage per day (once per day before bedtime) is described as being in the range 7.5 mg to 15 mg. It thus appears that, in view of its low and late bioavailability, the molecule is not suitable for administration in order to induce sleep rapidly in a subject having trouble sleeping, and a wait of 1 h to 2 h is not satisfactory in terms of therapeutic effectiveness. However, Doxylamine base or its salt, e.g. succinate, are easily soluble in ethanol, thereby making it possible, specifically by means of the invention, to facilitate starting sleep rapidly by causing drowsiness within a few minutes after administration via the oral mucous membrane in accordance with the invention. This enables subjects, who are often of the anxious type, to avoid having to wait for the late stage of sufficient bioavailability of the active principle when taken orally, and for the start of its effect. By means of the invention, they start sleeping in a time of a few minutes, and when it suits them, as a result of the almost-immediate bioavailability of the Doxylamine in the blood stream, as soon as it has been absorbed via the oral mucous membrane, instead of having to be patient, as previously, after taking a higher dose orally. The advantages of Doxylamine that have been known for a long time are that it is not the subject of drug-monitoring problems, and, above all, its rather-short half-life does not, on average, exceed the duration of a night's sleep (6 h to 9 h). This enables the subject to wake up without suffering from any persisting residual activity of the molecule continuing to induce drowsiness.

Administered via the oral mucous membrane in accordance with the invention, the useful dose for rapid sleep induction is not greater than a few milligrams, i.e. around 1 mg to 3 mg for an average useful dosage, with this naturally depending on the user's weight status, and on the user's particular sensitivity to the effect of Doxylamine.

Via the oral mucous membrane in accordance with the invention, Doxylamine reaches its H1 brain receptors without prior loss, before any systemic/organic distribution/dispersion (which is not the situation for orally-taken doses).

The useful dosages of the invention are thus substantially less than the dosages used orally (7.5 mg to 15 mg), and, above all, they are pharmacologically effective in a time that is reduced to a few minutes.

Furthermore, merely as a result of the “Flash” or “Bolus” characteristic associated with the concentrated vascular bioavailability of the oral mucous membrane form of the invention, which directs the dose towards the central nervous system directly via the arteries, the doses need be chosen prudently, as described in the corresponding examples below.

In the context of the present invention, the dosage of Doxylamine advantageously lies in the range 0.025 mg to 7 mg, preferably in the range 0.075 mg to 5 mg, and more preferably in the range 0.5 mg to 4 mg.

### Formulation A5: 1 mL for 4 mg of Doxylamine Succinate:

distilled water: 0.50 mL

ethanol: 0.50 mL

Doxylamine succinate: 4 mg

pH adjuster q.s. pH 6 to 8

### Formulation B5: 0.75 mL for 3 mg of Doxylamine Succinate:

distilled water: 0.375 mL

ethanol: 0.375 mL

Doxylamine succinate: 3 mg

pH adjuster q.s. pH 6 to 8

### Formulation C5: 0.5 mL for 2.5 mg of Doxylamine Succinate:

distilled water: 0.25 mL

ethanol: 0.25 mL

Doxylamine succinate: 2.5 mg

pH adjuster q.s. pH 6 to 8

### Formulation D5: 0.4 mL for 1.5 mg of Doxylamine Base:

distilled water: 0.20 mL

ethanol: 0.20 mL

Doxylamine base: 1.5 mg

pH adjuster q.s. pH 6 to 8

### Formulation E5: 0.3 mL for 0.75 mg of Doxylamine Base:

distilled water: 0.15 mL

ethanol: 0.15 mL

Doxylamine base: 0.75 mg

pH adjuster q.s. pH 6 to 8

### Formulation F5: 0.3 mL for 0.5 mg of Doxylamine Succinate:

distilled water: 0.15 mL

ethanol: 0.15 mL

Doxylamine succinate: 0.5 mg

pH adjuster q.s. pH 6 to 8

### 6—Cyproheptadine:

Cyproheptadine (MW=287 Da) is another lipophilic molecule, an H1-antihistamine having a central sedative effect, that is administered orally at 4 mg per unit, but it nevertheless possesses a half-life that is longer than the half-life of Doxylamine.

When taken orally, its peak plasma levels occur only 2 h to 3 h after ingestion, so Cyproheptadine can advantageously benefit from the invention in order to induce sleep rapidly.

Since this molecule has bioavailability of around only 25% of the dose administered orally, a formulation of the invention rapidly inducing sleep can advantageously be defined for Cyproheptadine, as shown below by way of non-limiting examples.

In the context of the present invention, the dosage of Cyproheptadine advantageously lies in the range 0.025 mg to 3 mg, preferably in the range 0.075 mg to 2.5 mg, and more preferably in the range 0.5 mg to 1 mg.

### Formulation A6: 0.5 mL for 1 mg of Cyproheptadine Base:

distilled water: 0.25 mL

ethanol: 0.25 mL

Cyproheptadine base: 1 mg

### Formulation B6: 0.3 mL for 0.5 mg of Cyproheptadine Base:

distilled water: 0.15 mL

ethanol: 0.15 mL

Cyproheptadine base: 0.5 mg

III—Sleep Induction and/or Resynchronization of the Circadian Rhythm:

### 7—Melatonin:

Melatonin or N-Acetyl-5-Methoxytryptamine is a brain hormone for adjusting the chronobiological rhythms and in practice most of the hormonal secretions in human beings. This neurohormone is synthesized from a neurotransmitter, serotonin, which is itself derived from tryptophan, an essential amino acid. It is secreted in the brain by the pineal gland in response to the absence of light. Microbes, various algae, and plants also produce Melatonin (“Phytomelatonin” in plants). The organism may also extract it from some of these plant sources (rice, bananas, pineapples, etc.). It adjusts the sleep cycle and the other circadian rhythms. Normally, the sleep hormone is secreted only at night time (peak secretion levels in human beings being at 5 O'clock in the morning because its production is inhibited by light), and it manages circadian rhythms (in part). Wake/sleep synchronization disorders mainly occur as a result of long-haul flights and they are associated with passing through multiple time zones, or they may occur in subjects who are subjected to night work over a long period of time.

Documents WO 00/72843, EP 0 518 468, WO 2007/099172, and EP 0 835 652 describe the use of Melatonin in medical and cosmetic treatments. Such applications are preferably in forms that can be administered via the digestive tract, or they are cosmetic compositions, all exempt of ethanol.

Melatonin is mainly administered orally, in the form of tablets or capsules, as a clinically-proven inducer of sleep, and also as a reducer for reducing sleep-access time, and finally for reestablishing wake/sleep synchronization. However, the bioavailability of the Melatonin taken orally is low, of the order of 15% of the ingested dose, and its half-life is short, scarcely greater than 50 min. It is a lipophilic molecule of low molecular weight (MW=232 Da) and very soluble in ethanol, and this makes it particularly suitable for use in formulations of the invention, since the stability of the Melatonin in alcohol solution increases with increasing degree of alcohol, starting from 35°. Via the oral mucous membrane, the formulations of the invention thus make it possible to administer a complete dose of Melatonin directly to the vascular system, which dose reaches its specific brain receptors in its entirety and in a few seconds, so as to act without delay to compensate the changes to the circadian rhythm, or so as to induce and facilitate access to sleep, and this is achieved with useful doses that are low. Specifically, is has been shown that when administered orally, high doses of Melatonin may even be counter-productive: several studies by the Massachusetts Institute of Technology (MIT) have shown that the Melatonin supplements that are available over the counter contain three to ten times more of this hormone than is necessary for exerting an activity on sleep, when administered orally. In the studies, the doses normally administered orally, at bedtime, varied over the range 0.3 mg to 5 mg. High doses of Melatonin may exert a negative paradoxical action that propagates to phases that are not phases of responding to Melatonin. In another study, 0.5 mg of melatonin taken orally was effective, but 20 mg (forty times more) was not. However, the bioavailability mechanisms of Melatonin, which has a short half-life, vary greatly from one individual to another, and they are degraded by digestive administration, which is not satisfactory for the best application or for obtaining the optimum effectiveness of Melatonin. This implies that Melatonin ought to be administered in a manner that is particularly suitable for enabling the dose that is right for the subject to be delivered in immediate and complete manner at night time and after 9 PM, so as to induce sleep rapidly, as specifically made possible by the invention. Finally, the compatibility of administrations of the invention is improved by the fact that, unlike a medication of the hypnotic type, Melatonin does not cause side effects after its sleep-inducing effect, nor does it cause phenomena of dependency.

The formulations of the invention mentioned in the examples are preferably established on the basis of a high degree of alcohol, i.e. about 45° to 50° of Ethanol, and this is in order to cause rapid and immediate absorption of all of the solution as soon as said solution is deposited in contact with a preferred mucous-membrane zone selected for this purpose, e.g. gum/cheek. As a result, by the particular means of the invention, it is possible to deliver a concentration of Melatonin into a small volume of circulating blood, with this “bolus” effect making it possible to increase the effectiveness of very low doses of Melatonin, since they reach their brain receptors without being subjected to an excessive dispersion phenomenon in the arterial blood stream that carries them. As a result of its lipophilic structure, not only is the small molecule of Melatonin absorbed in its entirety in preferred and rapid manner by the mucous surface on which it is deposited, but, above all, it can diffuse easily into the organism, in particular into the intracellular membranes, and thus, by means of the nanostructures of active principle of the invention, it can rapidly cross the blood-brain barrier, particularly so as to reach the neurons (synapses), whence its ability to act rapidly and effectively at low dosages on the central nervous system. The “bolus” effect corresponds to the effect that can be caused by “flash” intravenous injection of an active principle. The invention is thus particularly different from other forms of oral mucous membrane administration that do not have this “bolus” effect, in particular gel forms, or chewable-tablet forms, or tablets that disintegrate, or mucous-membrane adhesive forms, which slowly release the active principle, or “spray” forms, which disperse widely on contact with non-bounded surfaces of the oral mucous membrane, with, for all of the above-mentioned forms, very significant losses associated with inevitable reflex swallowing of a large portion of the dose that has been delivered.

In the context of the present invention, the dosage of Melatonin advantageously lies in the range 0.015 mg to 10 mg, preferably in the range 0.025 mg to 7.5 mg, and more preferably in the range 0.05 mg to 5 mg.

### Formulation A7: 0.5 mL for 5 mg of Melatonin Base:

distilled water: 0.25 mL

ethanol: 0.25 mL

Melatonin base: 0.5 mg

### Formulation B7: 0.5 mL for 3 mg of Melatonin Base:

distilled water: 0.25 mL

ethanol: 0.25 mL

Melatonin base: 3 mg

### Formulation C7: 0.5 mL for 2 mg of Melatonin Base:

distilled water: 0.23 mL

ethanol: 0.27 mL

Melatonin base: 2 mg

### Formulation D7: 0.5 mL for 1 mg of Melatonin Base:

distilled water: 0.255 mL

ethanol: 0.245 mL

Melatonin base: 1 mg

### Formulation E7: 0.3 mL for 0.8 mg of Melatonin Base:

distilled water: 0.15 mL

ethanol: 0.15 mL

Melatonin base: 0.8 mg

### Formulation F7: 0.3 mL for 0.5 mg of Melatonin Base:

distilled water: 0.15 mL

ethanol: 0.15 mL

Melatonin base: 0.5 mg

### Formulation G7: 0.3 mL for 0.3 mg of Melatonin Base:

distilled water: 0.15 mL

ethanol: 0.15 mL

Melatonin base: 0.3 mg

### Formulation H7: 0.3 mL for 0.1 mg of Melatonin Base:

distilled water: 0.15 mL

ethanol: 0.15 mL

Melatonin base: 0.1 mg

### Formulation I7: 0.25 mL for 0.05 mg of Melatonin Base:

distilled water: 0.13 mL

ethanol: 0.12 mL

Melatonin base: 0.05 mg

The invention also applies advantageously to pharmaceutical analogs of Melatonin, including, as non-limiting examples, Ramelteon and Agomelatine.

### 8—Ramelteon:

Although Melatonin is not widely recommended by medical authorities, Ramelteon ((S)—N-(2-(1,6,7,8-tetrahydro-2H-indeno-(5,4)furan-8-yl)ethyl)propionamide), sold under the trade name Rozerem (property of Takeda Pharmaceuticals), which is designed to activate the MT1 and MT2 receptors of Melatonin, has been approved for treating insomnia in the United States.

The invention represents a remarkable opportunity when applied to Ramelteon: specifically, this molecule, administered orally, is a lipophilic active principle, since 82% of it is bonded to plasma proteins and since it is of low molecular weight (MW=259.34 Da), with a short half-life of only 1 h to 2.6 h, and, above all, when administered orally having extremely low bioavailability of only 1.8% of the administered dose, established commercially at 8 mg and 16 mg of Ramelteon per unit.

It should thus be understood that this molecule, for which 98.2% of its orally-administered dose is destroyed, may benefit greatly from being administered via the oral mucous membrane as in the invention, which offers it immediate vascular bioavailability at very low useful dosages. What is more, this molecule does not generate pharmacological dependency, and does not have an addictive effect that leads to abuse, as with Benzodiazepines or the like. Finally, Ramelteon is completely suitable for formulations of the invention, since this small lipophilic molecule is very easily soluble in ethanol.

In the context of the present invention, the dosage of Ramelteon advantageously lies in the range 0.015 mg to 10 mg, preferably in the range 0.025 mg to 1.5 mg, and more preferably in the range 0.15 mg to 2.5 mg.

### Formulation A8: 0.3 mL for 0.15 mg of Ramelteon Base:

distilled water: 0.15 mL

ethanol: 0.15 mL

Ramelteon base: 0.15 mg

### Formulation B8: 0.3 mL for 0.3 mg of Ramelteon Base:

distilled water: 0.15 mL

ethanol: 0.15 mL

Ramelteon base: 0.3 mg

### Formulation C8: 0.3 mL for 0.4 mg of Ramelteon Base:

distilled water: 0.12 mL

ethanol: 0.18 mL

Ramelteon base: 0.4 mg

### Formulation D8: 0.4 mL for 0.5 mg of Ramelteon Base:

distilled water: 0.20 mL

ethanol: 0.20 mL

Ramelteon base: 0.5 mg

### Formulation E8: 0.4 mL for 1 mg of Ramelteon Base:

distilled water: 0.20 mL

ethanol: 0.20 mL

Ramelteon base: 1 mg

### Formulation F8: 0.5 mL for 1.5 mg of Ramelteon Base:

distilled water: 0.24 mL

ethanol: 0.26 mL

Ramelteon base: 1.5 mg

### Formulation G8: 0.5 mL for 2.5 mg of Ramelteon Base:

distilled water: 0.24 mL

ethanol: 0.26 mL

Ramelteon base: 2.5 mg

### 9—Agomelatine:

This is a small lipophilic molecule of low molecular weight (MW=243 Da), which presents a half-life that is less than 2 h and that is bioavailable at only less than 5% of an orally administered dose, which represents only 1.25 mg and 2.5 mg from doses usually administered at 25 mg and 50 mg respectively. The invention thus provides a significant advantage to the dose/effect ratio of this molecule, having a raw material price that exceeds US$3000 per kilogram, which is a significant saving in terms of dose and of cost for an application in which 95% of the active principle is lost as a result of it being administered orally.

In the context of the present invention, the dosage of Agomelatine advantageously lies in the range 0.015 mg to 5 mg, preferably in the range 0.025 mg to 3 mg, and more preferably in the range 0.8 mg to 2.5 mg.

### Formulation A9: 0.5 mL for 2.5 mg of Agomelatine Base:

distilled water: 0.25 mL

ethanol: 0.25 mL

Agomelatine base: 2.5 mg

### Formulation B9: 0.3 mL for 1.25 mg of Agomelatine Base:

distilled water: 0.15 mL

ethanol: 0.15 mL

Agomelatine base: 1.25 mg

### Formulation C9: 0.3 mL for 0.8 mg of Agomelatine Base:

distilled water: 0.15 mL

ethanol: 0.15 mL

Agomelatine base: 0.8 mg

In conclusion, it is useful to observe an additional and very significant advantage of the invention. It is known that many subjects use hypnotic active principles for suicide attempts. In view of their toxic potential at high doses, the molecules that are used the most are, by way of non-limiting example, mainly hypnotics forming part of the family of Benzodiazepines, or related molecules such as Cyclopyrrolones, Imidazopyridines, or Pyrazolopyrimidines. Since the invention enables rapid sleep induction for a very low dose of active principle, it can easily be understood that it becomes difficult for a subject who has taken a low and very rapidly effective dose via the oral mucous membrane in accordance with the invention, to repeat this administration successively enough times to administer a quantity of active principle that might cause suicide, since the invention causes the subject to fall asleep rapidly in just a few minutes after the initial administration, completely without toxicity. This is not the situation for a subject who accumulates and swallows all at once and in a few moments, a large number of high unit dose tablets available to the subject. The small useful dose and the comfort of falling asleep rapidly as provided by the invention are thus associated with much greater safety in terms of toxicology, both for users themselves and for the community as a whole.

Although the present invention is described above with reference to several examples, it should be understood that it is not limited to those examples, the scope of the invention being defined by the accompanying claims.

1. A dosage form for oral administration, via the mucous membranes, of an active principle for accelerated sleep induction and/or for treating sleep disorders and/or for treating a central nervous system disorder, the dosage form being characterized in that said active principle is lipophilic or amphiphilic and is in a dissolved state that is stable and complete in a hydro-alcoholic solution comprising 35% to 70% by weight of ethanol and 30% to 65% by weight of water, generating nanostructures of said active principle under consideration within the hydro-alcoholic solution, thereby enabling the active principle to cross the blood-brain barrier, said active principle being in base and/or salt form and belonging to the chemical families of sleep inducers or modulators of lipophilic or amphiphilic type and of molecular weight that is less than 1000 Da, said active principle being selected from the families of Imidazopyridines, including Zolpidem, the family of Cyclopyrrolones, including Eszopiclone, the family of Pyrazolopyrimidines, including Zaleplon, the family of Benzodiazepines, including Midazolam and Brotizolam, the family of H1-antihistamine sedatives, including Doxylamine and Cyproheptadine, and/or the family of Melatonin and Melatonin agonists, including Melatonin, Ramelteon, and Agomelatine, the volume of said hydro-alcoholic solution being less than or equal to 2 mL, and said active principle being present at a dosage that is less than or equal to 8 mg, all of said active principle being absorbed in transmucosal manner via the mucous membranes of the floor of the oral cavity, in particular via the gums/cheeks, via the gum area, via the cheeks, or under the tongue.

2. A dosage form according to claim 1, wherein the hydro-alcoholic solution comprises 40% to 65% by weight of ethanol and 35% to 60% by weight of water.

3. A dosage form according to claim 1, wherein the hydro-alcoholic solution contains a pH adjusting agent.

4. A dosage form according to claim 3, wherein said active principle contains a carbonyl-acid function, said hydro-alcoholic solution containing a pH adjusting agent and/or a sequestering agent.

5. A dosage form according to claim 1, wherein the hydro-alcoholic solution is made up exclusively of ethanol, water, and an active principle.

6. A dosage form according to claim 1, wherein all of said active principle is absorbed transmucosally in less than 10 s, advantageously in less than 6 s.

7. A dosage form according to claim 1, wherein the molecular weight of said active principle is less than 600 Da.

8. A dosage form according to claim 1, wherein the dosage of said active principle is less than 5 mg.

9. A dosage form according to claim 1, wherein the volume of hydro-alcoholic solution is less than or equal to 1 mL.

10. A dosage form according to claim 1, wherein said active principle [symbol]
